(19) United States
(12) Patent Application Publication
(10) Pub. No.: US 2021/0369526 A1
(43) Pub. Date: Dec. 2, 2021

(54) PATIENT LIFT

(71) Applicants: Ernesto Heraclito Weisson, Miami, FL (US); Agustin Andrade, Miami, FL (US); Gloria Andrade-Feraud, Miami, FL (US)

(72) Inventors: Ernesto Heraclito Weisson, Miami, FL (US); Agustin Andrade, Miami, FL (US); Gloria Andrade-Feraud, Miami, FL (US)

(21) Appl. No.: 16/882,958

(22) Filed: May 26, 2020

(51) Int. Cl. A61G 7/10; A61B 5/00

(52) U.S. Cl. CPC A61G 7/1019; A61B 5/704; A61G 7/1001

(57) ABSTRACT

The present invention relates to a patient lift for assisting a patient onto an examination table that includes: a lift plate positioned between a first handle assembly and a second handle assembly; a plurality of alignment rollers at each corner of the lifting plate, where the alignment rollers are adapted to vertically guide the lifting plate during use; an electrical actuator on each side of the patient lift within each handle assembly, wherein the electrical actuator engages the lifting plate; a power source associated with each electrical actuator; an actuator shaft, where the actuator shaft extends from the electrical actuator and is adapted to engage the lifting plate for the vertical movement of the lifting plate; and a base that provides support for the lifting plate when not in use. A first handle and a second handle extend from the base and are substantially u-shaped with vertical bars.

### BACKGROUND OF THE INVENTION

### Field of Invention

The present invention relates to a patient lift for assisting a patient onto an examination table.

### Description of Related Art

Individuals normally go to doctors for various examinations. Patients may present themselves with some form of ailment or physical limitation. Some physical issues can range from respiratory problems, pain, arthritis, or internal issues such as cardiovascular problems. Nonetheless, during an initial examination with a doctor, an individual normally has to use a step to get onto an examination table in a doctor's office. However, normally the step lacks any second- or third-degree point of balance, such as a hand rail, for the patient to easily control themselves while placing themselves on the examination table. Although many patients may be assisted by a nurse or other health care technician, it would be advantageous to have a system that is specifically designed to assist in lifting and allowing the patient to elevate vertically and therefore provide an easier means for sitting onto the examination table. Further, it would be advantageous to have a rail support associated with such a system.

### BRIEF DESCRIPTION OF DRAWINGS

FIG. 1 depicts a front view of a patient lift according to the present invention.

FIG. 2 depicts a second front view of the patient lift, where the lift platform is in a raised position.

FIG. 3 depicts a perspective view of the patient lift according to the present invention.

FIG. 4 depicts a second perspective view, where the platform is in a raised position.

### DETAILED DESCRIPTION

The present invention relates to a patient lift to assist a patient onto an examination table. The patient lift includes a lifting plate that is raised through the use of actuator electrical motors that enable the vertical movement of the lifting plate between two vertical handles. The vertical handles provide support for the patient as the patient steps onto the lifting plate for positioning onto an examination table. The lifting plate includes a controller that controls the vertical movement of the lifting plate and therefore a transfer of the lifting plate from a lower position to an upper position. Rollers are aligned with the handles that extend vertically from the base plate of the patient lift. A power source is provided on each side of the patient lift that powers the linear actuators that enable movement of the lifting plate.

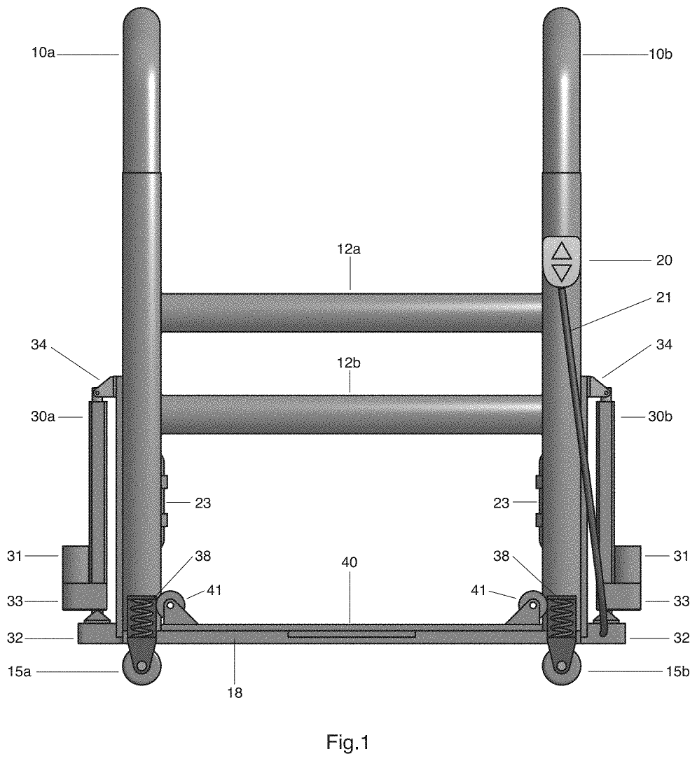

FIG. 1 depicts a front view of a patient lift according to the present invention. In this front view, a first handle 10a is found on a first side of the patient lift and a second handle 10b is found on a second side of the patient lift. Each handle 10a, 10b is substantially U-shaped extended from a base 18 of the patient lift. A first crossbeam 12a and a second crossbeam 12b are provided extending horizontally between the handles 10a, 10b. The crossbeams 12a, 12b are provided on a rear side of the patient lift. Other aspects depicted in FIG. 1 include the lifting plate 40 which is in a lowered position in FIG. 1 and alignment rollers 41 are shown that enable for a stable movement of the lifting plate 40 in a vertical plane. The patient lift is supported upon wheels 15a, 15b below a base 18. The wheels 15a, 15b engage a spring stabilizing system 38 on each handle 10a, 10b. A linear actuator is shown on each side that engages the lifting plate for lifting purposes. The linear actuators are shown as a first linear actuator 30a on a first side and a second linear actuator 30b on a second side. Each linear actuator 30a, 30b is powered by an electrical motor 31 shown at the lower end of each actuator 30a, 30b.

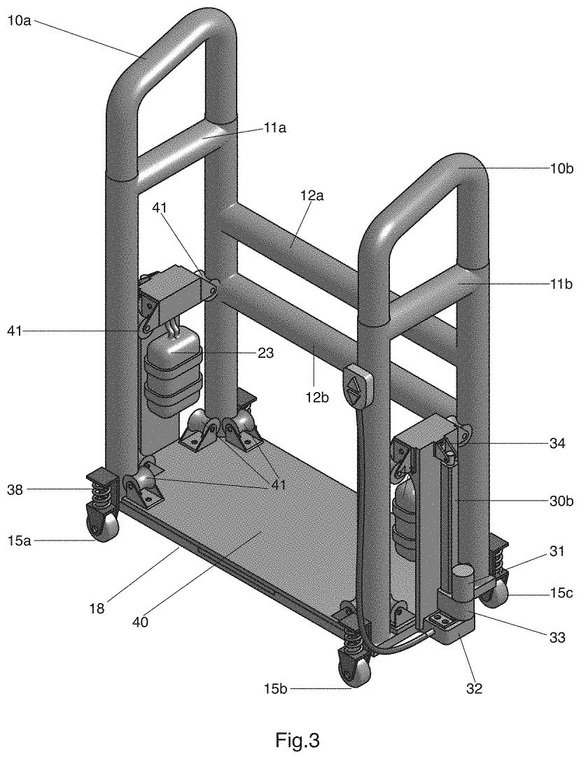

Further a gearbox 33, shown in FIG. 3, is provided that engages the motor 31 also on each side of the patient lift. Below the gearbox 33 is a linear actuator base attachment 32 that attaches the gearbox 33 and motor 31 to the base 18. The upper end engages the lifting device at an actuator lifting plate attachment site 34 shown on each side of the patient lift. In the lowered position the lifting plate 40 engages into the based 18 is shown in FIG. 1.

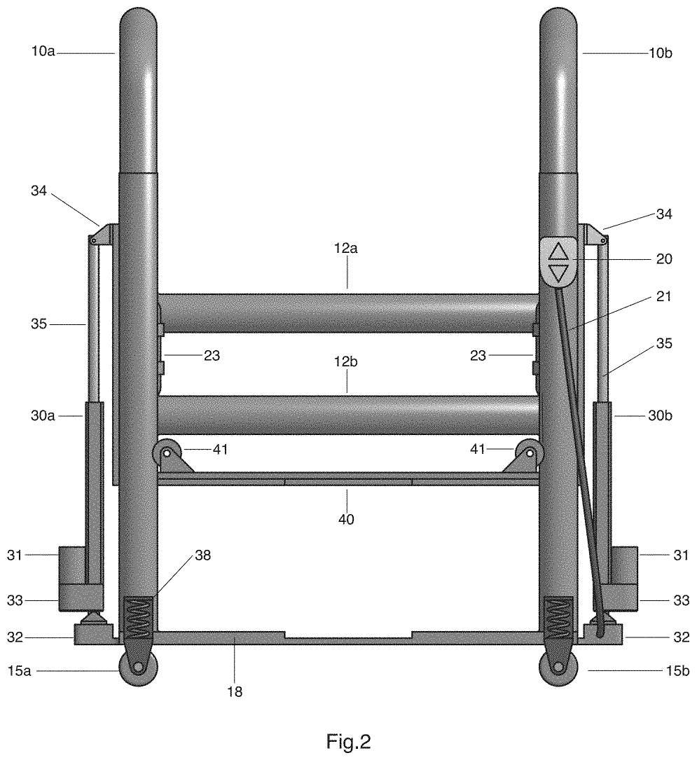

FIG. 2 shows the lifting plate 40 in a raised position, where it is lifted vertically and noted both actuators 30a, 30b are extended exposing an actuator shaft 35. In this extended position the actuators 30a, 30b enable the vertical movement of the lifting plate 40. A controller 21 shown on one side of handle 10b, where the controller 21 enables the movement of the actuators 30a, 30b. The controller 21 allows the user to move the lifting plate 40 in a vertical position as needed.

A perspective view of the patient lift is shown in FIG. 3. FIG. 3 provides a more detailed view of the alignment rollers 41 where a pair of alignment rollers are engaged with each vertical bar associated with each handle 10a, 10b. This positioning provides for a stable movement and firm engagement of the lifting plate during use. Also, this view shows a power source 23, which is provided for each actuator on each side of the patient lift. Further additional alignment rollers 41 are provided on the inside opening of each handle above the power source 23. Preferably the power source is a chargeable battery.

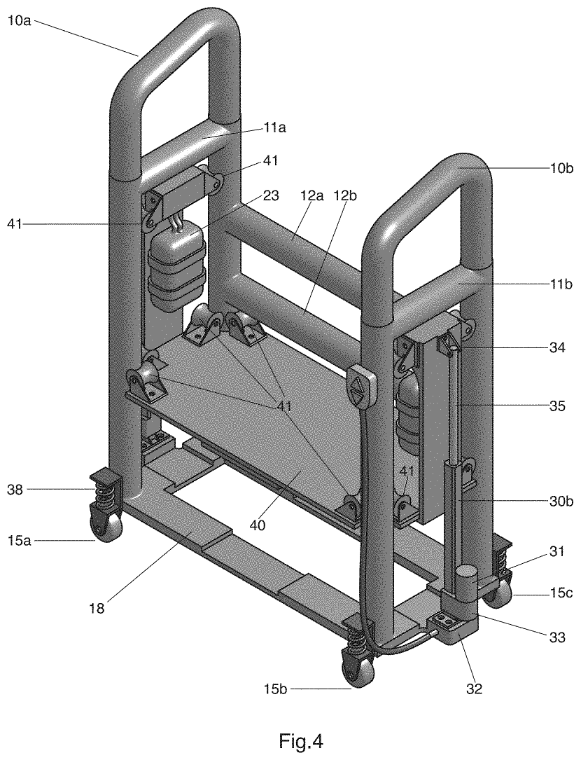

FIG. 4 shows the patient lift in an elevated position as opposed to the lowered position shown at FIG. 3. In this view, the alignment rollers 41 are more clearly seen in the engagement of the rollers 41 with the vertical bars of the handles 10a, 10b. Further crossbeams 11a, 11b are shown within each respective handle 10a, 10b, where the crossbeams 11a, 11b provide additional stability for the handles 10a, 10b. Each handle 10a, 10b extend vertically from the base 18 with stabilizing provided by the alignment rollers 41 and crossbeams.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A patient lift for assisting a patient onto an examination table comprising:
a lift plate positioned between a first handle assembly and a second handle assembly;
a plurality of alignment rollers at each corner of the lifting plate, where the alignment rollers are adapted to vertically guide the lifting plate during use;
an electrical actuator on each side of the patient lift within each handle assembly, wherein the electrical actuator engages the lifting plate;
a power source associated with each electrical actuator;
an actuator shaft, where the actuator shaft extends from the electrical actuator and is adapted to engage the lifting plate for the vertical movement of the lifting plate; and
a base that provides support for the lifting plate when not in use.

2. The patient lift according to claim 1, further including a first handle and a second handle, where the first handle and the second handle are u-shaped with vertical bars, wherein said vertical bars extend from the base.

3. The patient lift according to claim 2, further including at least one crossbeam extending horizontally between the first handle and second handle.

4. The patient lift according to claim 1, where the power source is a rechargeable battery.

5. The patient lift according to claim 1, where alignment rollers are provided on an actuator brace, where said alignment rollers engage the inside surface of the first handle and the second handle.

6. The patient lift according to claim 1, further including a controller, where said controller enables movement of the lift plate.
